(19) United States
(12) Patent Application Publication
(10) Pub. No.: US 2025/0099025 A1
(43) Pub. Date: Mar. 27, 2025

(54) BONE FUSION ASSESSMENT SYSTEMS AND METHODS

(71) Applicant: Providence Medical Technology, Inc., Pleasanton, CA (US)

(72) Inventors: Yu Suo, Las Vegas, NV (US); Nicholas Domek, Sacramento, CA (US); Christopher U. Phan, Dublin, CA (US); Shigeru Tanaka, Half Moon Bay, CA (US)

(21) Appl. No.: 18/730,472

(22) PCT Filed: Jan. 20, 2023
(86) PCT No.: PCT/US2023/011234

§ 371 Date: Jul. 19, 2024

Related U.S. Application Data
(60) Provisional application No. 63/301,074, filed on Jan. 20, 2022.
(60) Provisional application No. 63/415,090, filed on Oct. 11, 2022.

(51) Int. Cl. A61B 5/00; A61B 5/0538

(52) U.S. Cl. CPC A61B 5/4504; A61B 5/0538; A61B 5/6823; A61B 5/686

(57) ABSTRACT

An access instrument system that can identify tissue type and a bone fusion assessment system are disclosed. In one embodiment, the bone fusion assessment system includes a smart implant including: an implant body adapted to be affixed to a bone of a patient and to provide a therapeutic benefit thereto; and a bone fusion measurement system. The bone fusion measurement system includes a processing element; a sensor in electrical communication with the processing element and adapted to measure health data of the patient; and a wireless interface adapted to receive power and transmit the health data.

### CROSS-REFERENCE TO RELATED APPLICATIONS

This application claims priority to U.S. Provisional Patent Application No. 63/301,074, filed Jan. 20, 2022, entitled “BONE FUSION ASSESSMENT SYSTEMS AND METHODS”, and U.S. Provisional Patent Application No. 63/415,090, filed Oct. 11, 2022, entitled “BONE FUSION ASSESSMENT SYSTEMS AND METHODS”, each of which is incorporated by reference herein, in the entirety and for all purposes.

### FIELD

This application relates to devices and methods for measuring and/or assessing fusion of bones.

### BACKGROUND

Many surgical interventions involve the use of implants, cages, or other devices to support bones or bone portions. For example, plates may be used to couple two or more portions of a broken bone. Similarly, cages may be used to provide vertebral separation and fusion to treat degenerative disc disease of the spine. Positioning devices can be a challenge for surgeons because under traditional methods and systems there is limited to no feedback regarding the position of the access instrument (i.e., the instrument used to provide access to the portion in the body the device will be placed at). For example, a surgeon may not know what type of tissue the access instrument is in contact with. Due to these limitations in the field, surgeons often rely on fluoroscopy or other imaging technique for visualization. However, relying on a technique such as fluoroscopy exposes the patient to radiation. Improved methods and systems to provide feedback regarding the position of the access instrument is needed.

Bone fusion is one measure used to assess the success of a surgical intervention. For example, the fusion of two or more bone portions of a broken bone, two or more adjacent vertebrae, and/or fusion of an implant to a bone. Currently, no uniform or practical method or tool exists for measuring bony fusion including but not limited to spinal fusions. Some existing methods rely on radiographic interpretation of bone growth. For example, a radiologist or other medical provider may review post-operative imaging at progressively longer spans of time after a surgical intervention and estimate bone fusion (either between bones, bone portions, or bone and an implant). Such radiological methods are not accurate nor consistent, and expose patients to un-necessary radiation from x-rays, computed tomography (“CT”) scans, magnetic resonance imaging (“MRI”), and/or the like. Some other existing methods make use of secondary measurements of pain scores and neck motion and do not measure actual joint fusion. Improved methods and systems to measure and assess bone fusion in patients are needed.

### BRIEF SUMMARY

A bone fusion assessment system is disclosed. The system includes a smart implant including an implant body adapted to be affixed to a bone of a patient and to provide a therapeutic benefit thereto; and a bone fusion measurement system. The bone fusion measurement system includes a processing element, a sensor in electrical communication with the processing element and adapted to measure health data of the patient, and a wireless interface adapted to receive power and transmit the health data.

Optionally, in some embodiments, wherein the health data includes a physical characteristic selected from voltage, resistance and impedance which is interpreted to provide bone fusion data relating to fusion of the implant body to the bone of the patient, and the sensor is adapted to measure the bone fusion data.

Optionally, in some embodiments, the bone fusion assessment system includes an external device adapted to transmit the power to the bone fusion measurement system via the wireless interface.

Optionally, in some embodiments, the external device is further adapted to receive the health data via the wireless interface.

Optionally, in some embodiments, the bone fusion measurement system includes an internal power supply adapted to be charged from the external device via the wireless interface.

Optionally, in some embodiments, the bone fusion measurement system includes an internal power supply comprising a primary battery.

Optionally, in some embodiments, the sensor comprises at least one of a strain sensor, a piezo-resistive polymer, a capacitive transducer, a passive resonator, a surface acoustic wave resonator, an ultrasound emitter and detector, a galvanometer, a current sensor, a capacitance sensor, an inductance sensor, or an impedance sensor.

Optionally, in some embodiments, the strain sensor includes at least one of a piezoelectric sensor or Wheatstone bridge strain gauge.

Optionally, in some embodiments, the bone fusion data includes at least one of bone density, bone growth, a temperature change, an electrical property, a bone loading.

Optionally, in some embodiments, the bone loading includes at least one of a force, stress, strain, or pressure.

A bone fusion assessment system is disclosed. The system includes a wearable device adapted to be worn by a patient. The wearable device includes a bone fusion measurement system including: a processing element, a sensor in electrical communication with the processing element and adapted to measure health data of the patient, and a wireless interface adapted to receive power and transmit the health data.

Optionally, in some embodiments, the sensor includes at least one of a motion sensor, camera-based motion analyzer, a strain gauge, an impedance sensor, or an ultrasound transmitter/receiver.

Optionally, in some embodiments, the motion sensor comprises an accelerometer.

Optionally, in some embodiments, the health data includes at least one of a gait, a range of motion, excessive motion, a loading on the wearable device, a bone growth, or a mobility of the patient.

A method of assessing bone fusion of a patient is disclosed. The method includes supplying at least one of a smart implant or a wearable device to a patient. The smart implant or wearable device includes: an implant body adapted to be affixed to a bone of a patient and to provide a therapeutic benefit thereto; a bone fusion measurement system including: a processing element, a sensor in electrical communication with the processing element and adapted to measure health data of the patient, and a wireless interface adapted to receive power and transmit the health data.

Optionally, in some embodiments, the method includes powering the bone fusion measurement system via the wireless interface to cause the bone fusion measurement system to measure the health data.

Optionally, in some embodiments, the method includes receiving from the bone fusion measurement system the health data by an external device.

Optionally, in some embodiments, the method includes determining by the external device a fusion score based on the health data.

Optionally, in some embodiments, the method includes displaying the fusion score to a healthcare provider; and determining a treatment plan based on the fusion score.

Optionally, in some embodiments, the health data includes bone fusion data relating to fusion of the implant body to the bone of the patient, and the sensor is adapted to measure the bone fusion data.

Optionally, in some embodiments, the method includes at least one of a strain sensor, a piezo-resistive polymer, a capacitive transducer, a passive resonator, a surface acoustic wave resonator, an ultrasound emitter and detector, a galvanometer, a current sensor, a capacitance sensor, an inductance sensor, an impedance sensor, a motion sensor, or a camera-based motion analyzer.

An access instrument system is disclosed. The access instrument system includes an access instrument. The access instrument system includes a sensor system. The access instrument system includes a processing element in communication with the sensor system. The processing element may send power to the sensor system, receive current data from the sensor system, and calculate an impedance measurement based on the current data and the power sent to the sensor system. The access instrument system includes a physical component in communication with the processing element. The physical component may transmit the impedance measurement.

Optionally, in some embodiments, the sensor system includes a current carrying electrode. The current carrying electrode may receive the power sent by the processing element and may send current to a tissue area. The sensor system includes a current measuring electrode. The current measuring electrode may receive current from the tissue area and may generate the current data.

Optionally, in some embodiments, the sensor system includes a current carrying electrode disposed at a distal end of the access instrument. The sensor system includes a current measuring electrode disposed at the distal end of the access instrument. The current carrying electrode and the current measuring electrode are separated by at least one of an air gap and an insulator.

Optionally, in some embodiments, the sensor system includes a current carrying electrode encased in an insulating sleeve. The sensor system includes a current measuring electrode. The current measuring electrode may encase the insulating sleeve.

Optionally, in some embodiments, the sensor system includes a current carrying electrode. The current carrying electrode may contact a skin surface. The sensor system includes a current measuring electrode disposed at a distal end of the access instrument.

Optionally, in some embodiments, the sensor system includes a current carrying electrode disposed at a distal end of the access instrument. The sensor system includes a pair of current measuring electrodes disposed between a proximal end of the access instrument and the distal end of the access instrument.

Optionally, in some embodiments, the access instrument system includes a distal end of the access instrument. The distal end includes a first portion and a second portion, the first portion may be separated from the second portion. The sensor system includes a first current carrying electrode disposed at the first portion of the distal end of the access instrument. The sensor system includes a first current measuring electrode disposed at the second portion of the distal end of the access instrument. The sensor system includes a second current carrying electrode disposed between a proximal end of the access instrument and the distal end of access instrument. The sensor system includes a second current measuring electrode disposed between a proximal end of the access instrument and the distal end of access instrument, the second current measuring electrode separated from the second current carrying electrode.

Optionally, in some embodiments, the access instrument system includes a distal end with a chiseled tip. The chiseled tip may wedge inside a facet joint.

Optionally, in some embodiments, the access instrument system includes a body with at least one button. The button may alter a frequency of the current sent by the processing element.

Optionally, in some embodiments, the access instrument system includes a body comprising plastic. The access instrument system includes a tip comprising an insulating material.

An implant system is disclosed. The implant system includes an implant. The implant system includes a sensor system. The implant system includes a processing element in communication with the sensor system. The processing element may send power to the sensor system, receive current data from the sensor system, and calculate an impedance measurement based on the current data and the power sent to the sensor system. The implant system includes a physical component in communication with the processing element. The physical component may transmit the impedance measurement.

An implant system is disclosed. The implant system includes an implant. The implant system includes a sensor system. The implant system includes a processing element in communication with the sensor system. The processing element may measure one or more electrical properties selected from the list comprising: Charge (Q), Capacitance (C), Inductance (L), Voltage (V), Current (I), Resistance (omega), Power (P), Conductance (G), Impedance (Z), Frequency (f), and Amplitude (A) and calculate a change in the sensor system based on the one or more electrical properties. The implant system includes a physical component in communication with the processing element. The physical component may transmit the change in the system to measure fusion.

Optionally, in some embodiments, the sensor system includes a current carrying electrode. The current carrying electrode may receive the power sent by the processing element and may send current to a tissue area. The sensor system includes a current measuring electrode. The current measuring electrode may receive current from the tissue area and may generate the current data.

Optionally, in some embodiments, the sensor system includes a current carrying electrode disposed on an outer surface of the implant. The current carrying electrode may be in direct contact with a tissue area. The sensor system includes a current measuring electrode separated from the current carrying electrode.

Optionally, in some embodiments, the sensor system includes a current carrying electrode. The current carrying electrode may contact a skin surface. The sensor system includes a current measuring electrode disposed on an outer surface of the implant. The current measuring electrode separated from the current carrying electrode.

Optionally, in some embodiments, the implant may define an opening and the implant may be an interbody cage with a through surface and an outer side surface. The sensor system includes a current carrying electrode. The sensor system includes a current measuring electrode separated from the current carrying electrode. The current measuring electrode may be disposed on at least one of the through surface and the outer side surface.

Optionally, in some embodiments, the implant includes a top surface and a bottom surface. The sensor system includes a current carrying electrode. The sensor system includes a current measuring electrode separated from the current carrying electrode. The current measuring electrode may be disposed on at least one of the top surface and the bottom surface.

Optionally, in some embodiments, the processing element includes an internal power supply. The internal power supply may be a battery. The internal power supply may include a plurality of capacitors.

Optionally, in some embodiments, the implant system includes a sensor in communication with the processing element. The sensor may measure at least one of strain, pressure, load, and temperature.

A method of delivering an implant is disclosed. The method includes making an incision and exposing target bony elements. The method includes calculating, via a sensor system of an access instrument, a plurality of impedance measurements. The method includes identifying a target location based on the plurality of impedance measurements. The method includes inserting a tip of the access instrument into the target location. The method includes inserting an outer decorticator over the access instrument and decorticating the target location. The method includes inserting a guide tube over the access instrument, the guide tube to hold vertebrae apart. The method includes removing the access instrument through a shaft of the guide tube. The method includes inserting a decorticator rasp through the shaft of the guide tube and decorticating the target location. The method includes delivering the implant.

Optionally, in some embodiments, the identifying the target location includes calculating a difference in the plurality of impedance measurements and identifying a tissue type based on the difference.

Optionally, in some embodiments, the method includes inserting a decorticator burr through the guide tube prior to delivering the implant. The decorticator burr may decorticate articular surfaces of the target location.

Optionally, in some embodiments, the method includes activating the implant.

A method of activating an implant is disclosed. The method includes maintaining a processing element in a passive or semi-passive state. The processing element includes an internal power supply. The method includes producing power via the internal power supply. The method includes activating a sensor system in the implant by delivering the power produced by the internal power supply to the sensor system.

Optionally, in some embodiments, activating the sensor system includes receiving, via a current carrying electrode of the sensor system, the power produced by the internal power supply. Activating the sensor system includes sending, via the current carrying electrode, current to a tissue area. Activating the sensor system includes receiving, via a current measuring electrode of the sensor system, current from the tissue area. Activating the sensor system includes sending, via the current measuring electrode, current data to the processing element.

Optionally, in some embodiments, the processing element is communicably coupled with a physical component. The method further includes calculating, via the processing element, an impedance measurement based on the current data and the power produced by the internal power supply. The method further includes transmitting, via the processing element, the impedance measurement to the physical component. The method further includes transmitting, via the physical component, the impedance measurement.

Optionally, in some embodiments, the processing element is communicably coupled with a physical component and the internal power supply of the processing element comprises a plurality of capacitors. The method includes delivering power, via an external electromagnetic power source, to the physical component. The method includes charging the plurality of capacitors, via the physical component, with the power delivered by the external electromagnetic power source. The method includes producing power, via the plurality of capacitors.

### BRIEF DESCRIPTION OF THE SEVERAL VIEWS OF THE DRAWINGS

FIG. 1A is a simplified schematic representation of a bone fusion assessment system.

FIG. 1B is a simplified diagram of components of an example of a smart implant.

FIG. 1C is a simplified perspective view of an example of a smart implant positioned on an anterior cervical spine of a patient.

FIG. 1D is a simplified perspective view of an example of a smart implant positioned in a posterior cervical spine of a patient.

FIGS. 1E, 1F, 1G, 1H, 11, 1J, 1K, 1L and 1M illustrates various embodiments of a smart implant according to the present disclosure.

FIG. 2 is an example of a method of using a smart implant or wearable device according to the present disclosure.

FIG. 3A is a simplified schematic representation of an access instrument system.

FIG. 3B is a simplified diagram of components of an example of an access instrument.

FIG. 3C-1 is a simplified perspective view of an access instrument.

FIGS. 3C-2, 3C-3 and 3C-4 illustrate various embodiments of an access instrument according to the present disclosure.

FIG. 4 illustrates a method of delivering an implant.

FIG. 5 illustrates a method of activating an implant.

### DETAILED DESCRIPTION

Systems and methods of assessing bone fusion in a patient are disclosed. The disclosed systems include an access instrument that can identify tissue type and an implant, such as a smart implant, that includes a bone fusion measurement system. The implant may include a sensor that can measure patient health data such as bone fusion or other bone properties directly or indirectly. In some embodiments, a bone fusion measurement system is included with a wearable device that is not implanted in the body of the patient. The bone fusion measurement system includes a processing element that can receive the patient health data (e.g., strain, pressure, impedance, temperature, motion) from the sensor and transmit the same to a device external to the patient's body via a wireless interface. The smart implant can be self-contained and implanted in vivo. In some embodiments, the smart implant can remain in the patient's body for the effective life of the implant. In some embodiments, the smart implant is powered by an internal power source such as a battery or capacitor. In some embodiments, an external power source such as radio frequency or magnetic induction device, can power the smart implant wirelessly. The smart implant may use the external power supply to power the sensor, processing element and other components to evaluate and record objective measurements of the behavior of the implant, such as bone fusion. Implant data may be interpreted and transmitted back outside the patient's body to a receiver for display, interpretation, further analysis, transmission, and recording.

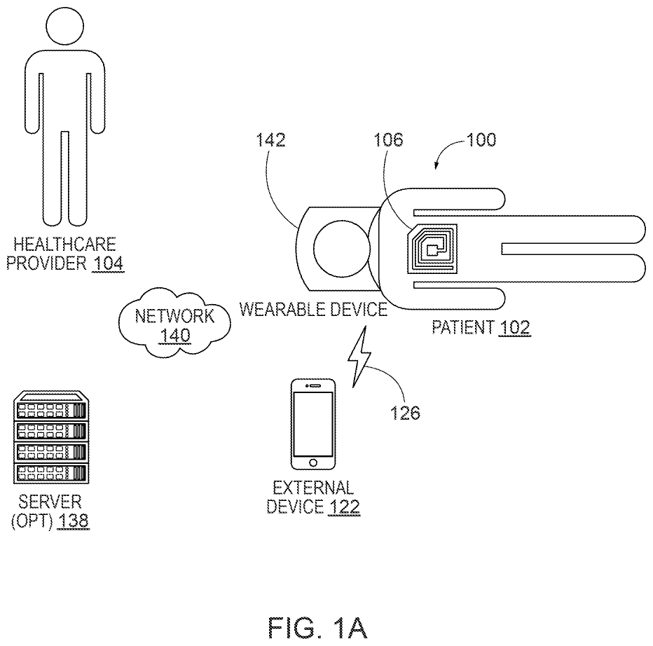

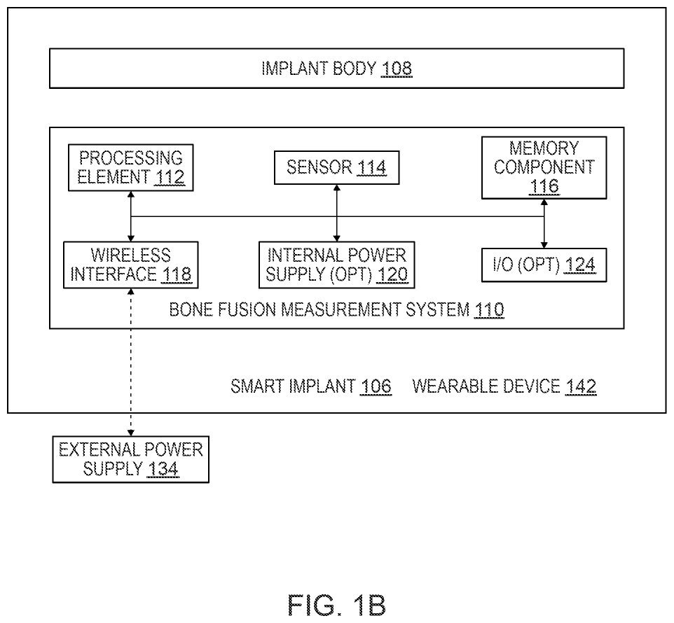

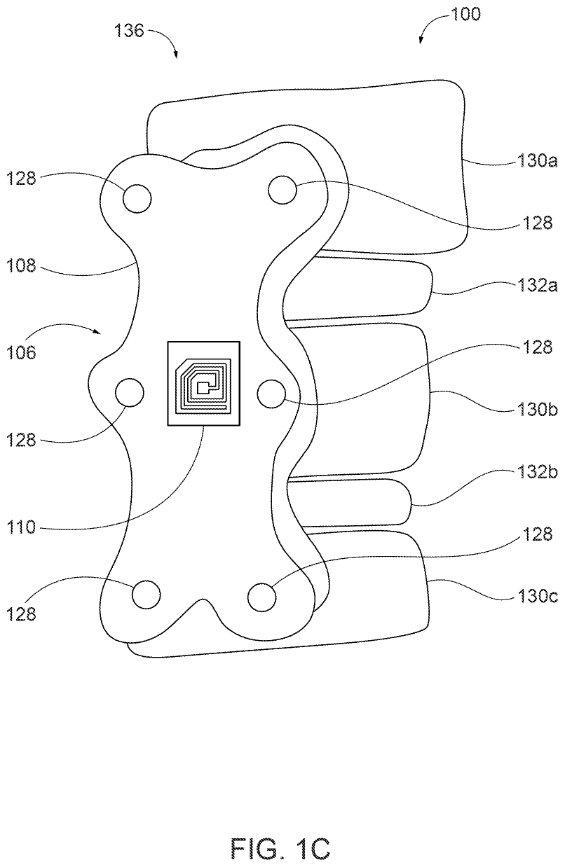

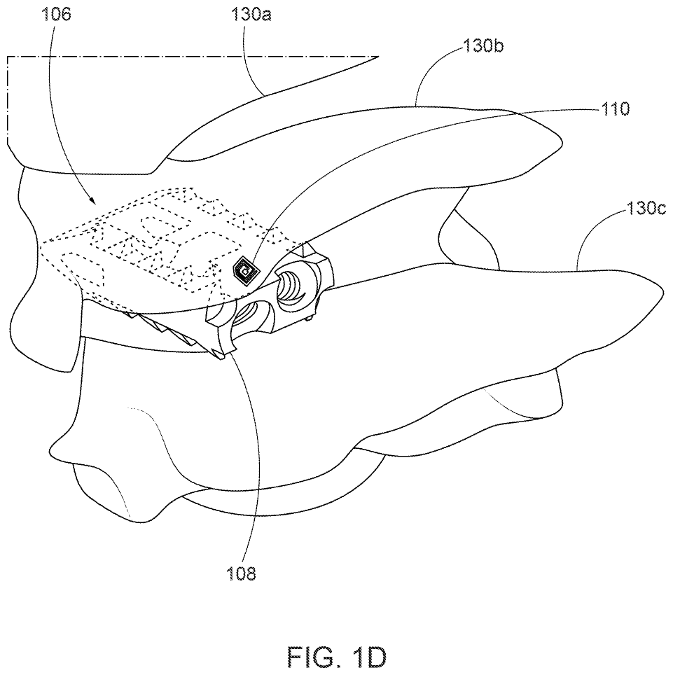

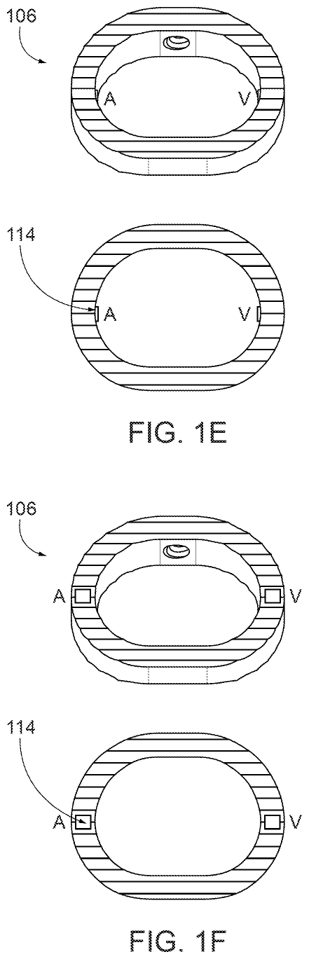

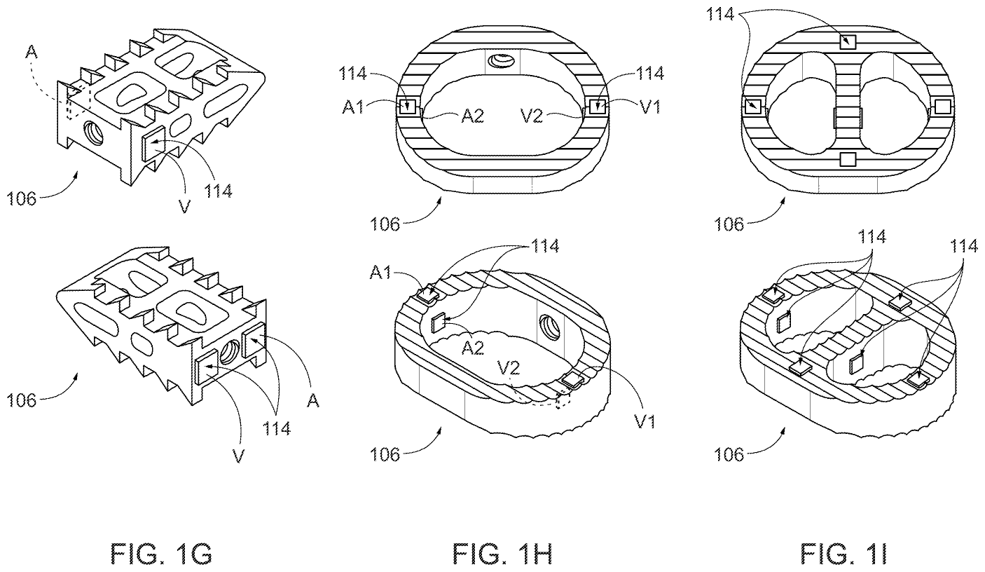

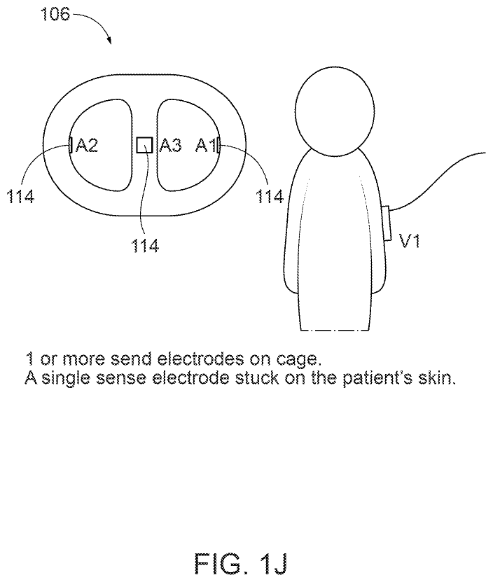

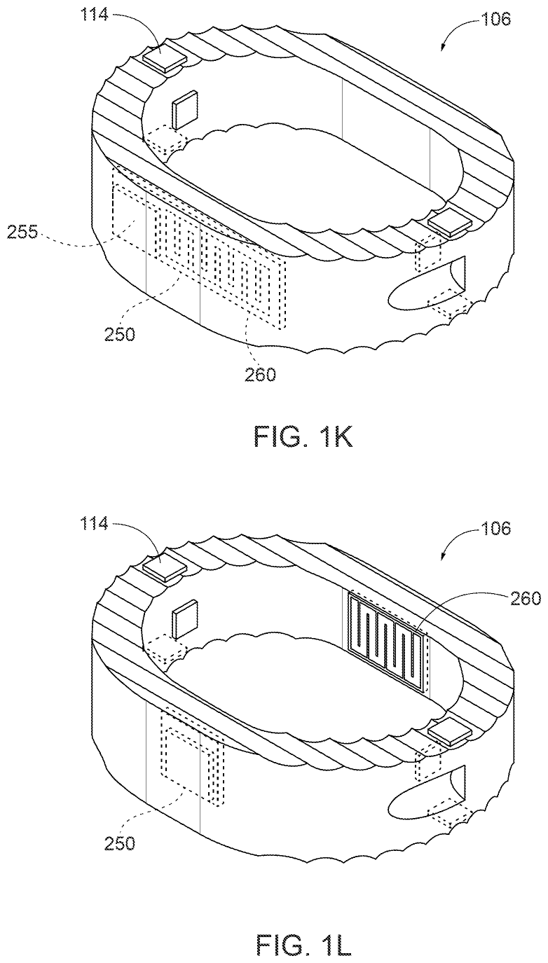

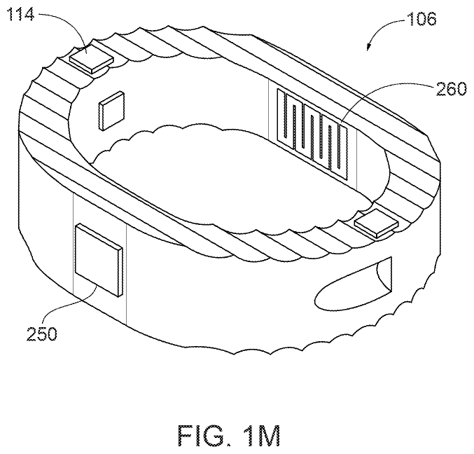

With reference to FIG. 1A, a bone fusion assessment system 100 is disclosed. The bone fusion assessment system 100 may also be referred to herein as an implant system 100. The bone fusion assessment system 100 may include a smart implant 106 and/or a wearable device 142 and an external device 122. The smart implant 106 and/or wearable device 142 includes a bone fusion measurement system 110 that measures health data including, but not limited to, bone fusion data. The bone fusion assessment system 100 may optionally include a network 140 and/or server 138. A healthcare provider 104 such as a doctor, surgeon, or the like may perform a surgical intervention on a patient 102. During the intervention, the healthcare provider 104 may install or position a smart implant 106 in the body of the patient 102. Alternately or additionally, the patient 102 may be fitted with a wearable device 142. In many implementations, the smart implant 106 may be coupled or fixed to a bone or bony aspect of the body of the patient 102. In some examples, the smart implant 106 may be fixed to the bone via a fastener 128 such as a screw, bolt, plate, pin, serration, or the like. The smart implant 106 may be further fixed to the bone of the patient 102 by a bone graft (e.g., an allograft or an autograft). The smart implant 106 may be fixed to the bone by a bone growth medium. As the patient 102 heals after the intervention, the bone of the patient may fuse to the smart implant 106. Additionally or alternately, portions of a bone may fuse to one another (e.g., portions of a bone on either side of a fracture), or a graft may fuse to a bone and/or smart implant 106.

FIG. 1B is a simplified diagram of components of an example of a smart implant 106. An external device 122 and/or server 138 may also have similar components as described with respect to FIG. 1B, but without the sensor 114. As also shown in FIG. 1B, a wearable device 142 may include a bone fusion measurement system 110 similar to that of the smart implant 106. When a wearable device 142 is used, the bone fusion measurement system 110 may be coupled to or integrated with the wearable device 142 outside the body of the patient 102. An implant with an implant body 108 may still be used in such cases but may be a “dumb” implant without a bone fusion measurement system 110.

The smart implant 106 includes an implant body 108, also referred to herein as an implant 108. The implant body 108 may be any device or structure suitable to provide a therapeutic effect to the patient 102 and which can be installed in vivo in the body of the patient 102. For example, the implant 108 may provide mechanical stabilization of the bones that are targeted for fusion. The implant body 108 may also be any device suitable to also support and/or receive a bone fusion measurement system 110. For example, an implant body 108 may be a plate, cage, screw, pin, collar, or other device that provides orthopedic support, constraint, separation, or other therapeutic effect to the patient 102. See, e.g., the example implant bodies 108 discussed herein with respect to FIG. 1C and FIG. 1D. In some embodiments, the implant body 108 may be a posterior cervical fusion (“PCF”) cage, an anterior interbody cage, plates (spine or extremities), pedicle screw/rod system, or the like. For example, the implant 108 may be one of a lumbar cage and a cervical spine cage. The implant may be or may include biocompatible material. For example, the implant 108 may include at least one of polyetheretherketone (PEEK), titanium, and three-dimensionally printed plastic, or a combination thereof. The implant body 108 may be suitable for use on any part of a body of a patient 102 such as the spine, hip, knee, leg, ankle, foot, shoulder, elbow, arm, wrist, hand, or skull. An implant body 108 may be placed in any suitable location on a bone where the implant body 108 may have a therapeutic effect e.g., back/proximal location, top/bottom surface, sidewall, or the like.

The implant 108 may include or define an outer surface. The outer surface of the implant 108 may be in contact with a tissue area. For example, components of the bone fusion measurement system 110 may be disposed on or in the outer surface and be in direct contact with the tissue area. The outer surface of the implant 108 may further define or include a top surface, a bottom surface, and side surfaces. For example, components of the bone fusion measurement system 110 may be disposed on or in the top surface, the bottom surface, and/or the side surfaces of the implant 108. The implant 108 may define an opening and thus may include a through surface and an outer side surface. Components of the bone fusion measurement system 110 may be disposed on or in the through surface and/or the outer side surface of the implant 108. Additionally, materials may be disposed within the opening of the implant 108. For example, materials such as biologics, bone graft, or the like may be included in the opening to promote and/or stimulate fusion.

As shown in FIG. 1B, the various devices of the bone fusion measurement system 110, external device 122, and/or server 138 may include one or more processing elements 112, one or more sensors 114, one or more memory components 116, a wireless interface 118, an optional internal power supply 120, and an optional input/output interface 124, where the various components may be in direct or indirect communication with one another, such as via one or more system buses, contract traces, wiring, or via wireless mechanisms.

The sensor 114 may be any device suitable to measure health data of a patient 102 such as bone fusion or other bone properties. By way of non-limiting example, the sensor 114 may measure motion (e.g., micro-motion, flex, looseness) of the implant body 108 or relative motion between the implant body 108 and/or one or more bone portions. An implant body 108 installed on a back/proximal location may be particularly suited to micro-motion measurement. Generally, the greater the relative motion, the less complete the fusion of the implant body 108/bone portions. Relative motion below a threshold, or absence thereof, may indicate suitable fusion of a bone, bone portions, and/or implant body. The sensor 114 may measure motion relative to a reference point (e.g., a surface of a joint).

The one or more sensors 114 may be a component of a sensor system. For example, the one or more sensors 114 may be a current carrying electrode and a current measuring electrode. For example, the current carrying electrode may receive power sent by the processing element 112, discussed herein, and may send current to a tissue area. The current measuring electrode may receive current from the tissue area and may generate current data. Thus, the current measuring electrode may be separated from the current carrying electrode, and vice-versa. For example, the current carrying electrode may be disposed on the outer surface of the implant 108 and may be in direct contact with the tissue area, and the current measuring electrode may be separated from the current carrying electrode by insulation or an air gap. Further, the current carrying electrode does not need to be disposed in or on the implant 108. For example, the current carrying electrode may contact a skin surface and the current measuring electrode may be disposed on the outer surface of the implant 108. The current measuring electrode may be disposed on any outer surface of the implant 108, e.g., the top surface, the bottom surface, the side surfaces, and/or the through surface and outer side surface in the case the implant 108 is a cage that defines the opening.

The one or more processing elements 112 may be disposed in or on the implant 108. The one or more processing elements 112 may be substantially any electronic device capable of processing, receiving, and/or transmitting instructions. For example, the processing elements 112 may be a microprocessor, microcomputer, graphics processing unit, advanced reduced instruction set computing (“RISC”) machine (“ARM”) chip, system on a chip, or the like. The one or more processing elements 112 may be the same or different geometric structures and may be two-dimensional or three-dimensional. For example, the processing element 112 may be a printed circuit board (PCB) of rectangular, circular, oval, and/or triangular shape. Furthermore, the one or more processing elements 112 may be constructed using various methods such as a single-sided, a double-sided, flexible, or even multi-sided.

The processing element 112 may include an internal power supply, as discussed herein. For example, the processing element 112 may include one or more batteries or at least one capacitor. For example, the processing element 112 may include at least enough capacitors to temporarily deliver enough power to activate the sensor system such that the current measuring electrode may make a single reading before discharging completely. In this example, the smart implant 106 may be “passive” when not in use, which reduces the risk of electrical shock and interference with other devices.

It also should be noted that the processing elements 112 may include one or more processing elements or modules that may or may not be in communication with one another. For example, a first processing element 112 may control a first set of components of the bone fusion measurement system 110 and a second processing element 112 may control a second set of components of the bone fusion measurement system 110 where the first and second processing elements 112 may or may not be in communication with each other. Relatedly, the processing element 112 may be configured to execute one or more instructions in parallel locally, and/or across a network 140, such as through cloud computing resources such as the optional server 138.

The processing element 112 may be in communication with the sensor system. For example, the processing element 112 may send power to the sensor system and may receive current data from the sensor system. The processing element 112 may calculate an impedance measurement based on the current data and the power sent to the sensor system. For example, the processing element 112 may send power to the sensor system and the current carrying electrode may receive the power sent by the processing element 112 and may send current to the tissue area. The current measuring electrode may receive current from the tissue area and may generate current data. The processing element 112 may receive the current data from the current measuring electrode and may calculate an impedance measurement corresponding to the current sent from the current carrying electrode and the current received by current measuring electrode.

The memory component 116 stores electronic data that may be utilized by the bone fusion measurement system 110, such as audio files, video files, document files, programming instructions, historical sensor data, implant configuration, and the like. The memory component 116 may be, for example, non-volatile storage, a magnetic storage medium, optical storage medium, magneto-optical storage medium, read only memory, random access memory, erasable programmable memory, flash memory, or a combination of one or more types of memory components.

The wireless interface 118 may also be referred to herein as a physical component 118. The wireless interface 118 receives and transmits data and/or power to and from the bone fusion measurement system 110 and the external device 122 or other devices of the bone fusion assessment system 100, such as a second processing element. See, e.g., the wireless communication/charging 126 shown schematically in FIG. 1A. For example, the physical component 118 may be in communication with the processing element 112. The physical component 118 may receive the impedance measurement from the processing element 112 and the physical component 118 may also transmit the impedance measurement, as discussed herein.

The wireless interface 118 may transmit data, such as the impedance measurement, to the external device 122 directly or indirectly. For example, the wireless interface 118 may transmit data to and from other computing devices through the network 140 or directly from device to device. In some embodiments, the wireless interface 118 may also include various modules, such as an application program interface (API) that interfaces and translates requests across the network 140 to external device 122. The wireless interface 118 may be any suitable wired or wireless interface. For example, the wireless interface 118 may include Wi-Fi, Bluetooth, Wi-Max, Zigbee, near field communication (“NFC”), radio frequency identification (“RFID”), a passive integrated transponder (“PIT”), cellular data, or the like.

The physical component 118 may be any shape. For example, the physical component 118 may two-dimensional or three-dimensional geometric shapes, such as a rectangular or a circular pattern. The physical component 118 may be communicatively coupled with the processing element 112. The physical component 118 may be directly attached to the processing element 112, external to the processing element 112 and internal to the implant 108, and/or external to the processing element 112 and external to the implant 108. For example, the processing element 112 may be a rectangular two-dimensional PCB embedded within the implant 108 with the physical component 118 disposed on one side of the PCB and electronic components disposed or included on the other side of the PCB. In this example, no wire needs to be routed outside of the PCB, and the physical component 118 does not need other fixation to the implant 108. In another example, the processing element 112 may be a rectangular two-dimensional PCB embedded within the implant 108 with electronic components on either or both sides of the PCB and the physical component 118 disposed on the outer surface of the implant 108 and connected to the PCB via a wire. In yet another example, both the processing element 112 and the physical component 118 may be disposed or attached to the outer surface of the implant 108. In this example, the processing element 112 may be flexible or curved to minimize the profile of the implant 108. The physical component 118 may or may not be attached directly to the processing element 112. The processing element 112 and the physical component 118 may be protected by an additional layer of material, such as plastic, PEEK, or titanium.

The wireless interface 118 may provide either, or both, data transmission/receipt and power to the bone fusion measurement system 110. For example, the physical component 118 may transmit the impedance measurement to the external device 122, also referred to herein as a second processing element 112. The second processing element 112 may store impedance values and/or measurements and calculate a difference between and/or in the stored impedance values and/or measurements. Additionally, the wireless interface 118 may be or may include an antenna that receives electromagnetic energy from an external device 122 used to power the bone fusion measurement system 110 without the bone fusion measurement system 110 using an internal power supply 120, or the electromagnetic energy from the external device 122 may be used to charge the internal power supply 325. For example, the physical component 118 may transmit power from the external device 122 to the processing element 112. For example, the external device 122 may include an external power supply 134 such as a radio frequency or magnetic induction antenna or coil that induces the flow of an electric current in the wireless interface 118 when the external power supply 134 and the wireless interface 118 are in proximity to one another. In some examples, the external device 122 is electromagnetic such that power may be transferred between the wireless interface 118 and the external device 122 by a magnetic field using inductive coupling (e.g., resonant inductive coupling) between coils of conductors in the wireless interface 118 and the external device 122, such as when the wireless interface 118 supports RFID. For example, the external power supply 134 may generate a time-varying electromagnetic field, which transmits power across space to the wireless interface 118, which extracts power from the electromagnetic field and supplies it to an electrical load in the bone fusion measurement system 110. In some examples, power may be transferred by using electric fields using capacitive coupling between conductive electrodes. In some examples, the external power supply 134 may transmit power to the wireless interface 118 via magnetodynamic coupling via one or more rotating magnets, microwaves, visible light waves, or other portions of the electromagnetic spectrum, etc. In some embodiments, separate external devices 122 may be used to separately power the bone fusion measurement system 110 and transmit/receive data. In some examples, the external device 122 may be a smart phone, smart watch, tablet computer, or the like. In some examples, data transmission between the external device 122 and the bone fusion measurement system 110 may be encrypted, such as to protect patient privacy.

The wireless interface 118 may receive power from the external power supply 134 and transmit data to the external device 122 simultaneously or in series. In some implementations, the external power supply 134 may provide power to the bone fusion measurement system 110 via the wireless interface 118 and the bone fusion measurement system 110 may charge an internal power supply 120 if used. For example, the external device 122 may deliver enough power to the passive implant 108 within a certain amount of time (e.g. about 30 seconds). Thus, the bone fusion measurement system 110 may be able to measure sensor data and/or store sensor data when the external power supply 134 is not present. The external device 122 may also provide heat therapy for the area surrounding the smart implant 106, such as the area surrounding the cervical or lumbar spine. The external device 122 may also provide range of motion measurements for the targeted fusion area, such as the cervical or lumbar spine. The external device 122 may also provide electromagnetic stimulation (e.g., may act as a bone growth stimulator) to the targeted fusion area, such as the cervical or lumbar spine.

The various devices of the system may optionally include an internal power supply 120. The internal power supply 120 provides power to various components of the bone fusion measurement system 110 or the external device 122. The internal power supply 120 may include one or more rechargeable, disposable, or hardwire sources, e.g., batteries, capacitor, dual layer capacitor (i.e., super capacitor) power cord, AC/DC inverter, DC/DC converter, or the like. In some embodiments, where the internal power supply 120 is a battery, the battery may be a rechargeable (i.e., secondary) battery or may be a single-use non-rechargeable (primary) battery. When a primary battery is used, the battery life may be long enough for the useful life of the implant body 108. Additionally, the internal power supply 120 may include one or more types of connectors or components that provide different types of power to the bone fusion measurement system 110. In some embodiments, the internal power supply 120 may include a connector (such as a universal serial bus) that provides power to the bone fusion measurement system 110 or batteries bone fusion measurement system 110 or external device 122 and also transmits data to and from the device to other devices.

The optional input/output interface 124 allows the bone fusion measurement system 110 and/or external device 122 to receive input from a user and provide output to a user. For example, the input/output interface 124 may include a capacitive touch screen, keyboard, mouse, stylus, or the like. The type of devices that interact via the input/output interface 124 may be varied as desired. The input/output interface 124 provides an input/output mechanism for devices of the bone fusion assessment system 100, such as to display visual information (e.g., images, graphical user interfaces, videos, notifications, and the like) to a user such as a healthcare provider 104, and in certain instances may also act to receive user input (e.g., via a touch screen or the like). The display may be an LCD screen, plasma screen, LED screen, an organic LED screen, or the like. The type and number of displays may vary with the type of devices (e.g., smartphone versus a desktop computer). In some example, the I/O interface may include an interface to the network 140, such as Wi-Fi, Bluetooth, an Ethernet socket, or the like. In some embodiments, data transmission from the sensor 114 to the external device 122 may be via the input/output interface 124 instead of, or in addition to, the wireless interface 118. For example, the external device 122 may communicate to the bone fusion measurement system 110 via wired communication, e.g., a device connected to the skin surface of the patient, or via needle access to the smart implant 106.

In another example, the sensor 114 may measure loading (i.e., force, stress, strain, and/or pressure) on the implant body 108. An implant body 108 implanted on a top/bottom or side portion of a bone may be particularly suited to measure loading. A reduction of loading below a threshold, or stable loading, may indicate suitable fusion of a bone, bone portions, and/or implant body. In some examples, loading may change during motion of the patient's body, e.g., during motion of a sliding joint. Measuring loading on the implant body 108 may be achieved without measuring a reference point. In another example, the sensor 114 may measure bone growth and/or bone density. For example, the sensor 114 may measure bone density, growth or other characteristics with ultrasound and/or changes in electrical properties (e.g., resistance, capacitance, and/or inductance) of the bone. In another example, the sensor 114 may measure changes in temperature of a bone or the implant body 108. For example, loading of the implant body 108 by a bone may cause changes in temperature of the bone and/or implant. Temperature changes below a threshold may indicate suitable fusion of a bone, bone portions, and/or implant body.

When a sensor 114 is used with a wearable device 142, the sensor may measure health data such as gait, range of motion (e.g., neck range of motion), excessive motion, loading on the wearable device 142, bone growth, patient 102 mobility, or the like.

The sensor 114 may use any suitable sensor technology that can measure relevant health data such as a strain sensor (e.g., a piezoelectric sensor or Wheatstone bridge strain gauge), a piezo-resistive polymer, a capacitive transducer, a passive resonator, a Surface Acoustic Wave (“SAW”) resonator, ultrasound emitter and/or detector, a galvanometer, current sensor, capacitance sensor, inductance sensor, impedance sensor, motion sensor (e.g., accelerometer), camera-based motion analyzer, or the like. Any sensor or combination of sensors may be used in either a smart implant 106 or a wearable device 142.

The bone fusion measurement system 110 may also include an analog to digital conversion device, which may be part of the sensor 114, processing element 112, or may be a separate device. The analog to digital conversion device may receive an analog signal from a sensing element in the sensor 114 and convert the same to a digital signal for further processing by the processing element 112, storage in the memory component 116, and/or communication to the external device 122 via the wireless interface 118.

The processing element 112 may execute an algorithm or firmware (stored for example on the memory component 116), to reject over-sensitivity, false positives, errors, or excess information received from the sensor 114. The bone fusion measurement system 110 may have a novel layout allowing for miniaturization including components like antennas, capacitors, flexible circuit boards, other novel design and layouts.

In some aspects, the processor, that assesses fusion, could be outside the body. That is, on the implant itself, it could just be providing a reading (eg: voltage, or impedance/resistance), for interpretation outside of the implant, post-wireless transmission to a handheld, or other device that interprets the data.

For example, the smart implant 106 illustrated in FIG. 1C has an implant body 108 in the form of a plate suitable to be affixed to the spine 136 (e.g., the anterior cervical spine 136) of the patient 102 to fuse two or more vertebrae together. The implant body 108 is secured to one or more vertebrae 130a, b, and c via one or more fasteners 128 such as screws. For example the implant body 108 may provide a therapeutic effect of fusing the vertebrae 130a-c to treat degeneration of the discs 132a,b between the vertebrae 130a-c. The sensor 114 may measure any type of data described above to assess the fusion of the implant body 108 to the cervical spine 136 of the patient 102.

In another example, the smart implant 106 illustrated in FIG. 1D includes an implant body 108 in the form of a cage, such as a cervical cage, suitable to treat degenerative disc disease of the spine 136. The implant body 108 includes a bone fusion measurement system 110 with a sensor 114 suitable to measure fusion of the implant body 108 to the vertebra 130b and vertebra 130c as discussed above.

In another example, the smart implant 106 illustrated in the perspective and top views of FIG. 1E includes an implant body 108 in the form of a cage, such as a lumbar interbody cage. The implant body 108 includes a bone fusion measurement system 110 with a sensor 114 suitable to measure fusion of the implant body 108 to the vertebra 130b and vertebra 130c as discussed above. The sensor 114 comprises impedance electrodes positioned in the inner ring of the implant, such as at positions A and V. Alternatively, and as shown in FIG. 1F, the impedance electrodes may be positioned on the top and bottom surfaces of the implant body, such as at positions A and V.

In another example, the smart implant 106 illustrated in FIG. 1G includes an implant body 108 in the form of a cage, such as a cervical cage. The implant body 108 includes a bone fusion measurement system 110 with a sensor 114 suitable to measure fusion of the implant body 108 to the vertebra 130b and vertebra 130c as discussed above. The sensor 114 comprises impedance electrodes positioned on the sides and rear (proximal) face of the implant, such as at positions A and V.

In another example, the smart implant 106 illustrated FIG. 1H includes an implant body 108 in the form of a cage, such as a lumbar interbody cage. The implant body 108 includes a bone fusion measurement system 110 with a sensor 114 suitable to measure fusion of the implant body 108 to the vertebra 130b and vertebra 130c as discussed above. The sensor 114 comprises impedance electrodes positioned in the inner ring and on one or more outer surfaces of the implant, such as at positions A1, A2 and V1, V2.

In another example, the smart implant 106 illustrated FIG. 1I includes an implant body 108 in the form of a cage, such as a lumbar interbody cage. The implant body 108 includes a bone fusion measurement system 110 with a sensor 114 suitable to measure fusion of the implant body 108 to the vertebra 130b and vertebra 130c as discussed above. The sensor 114 comprises impedance electrodes positioned in the inner rings and on one or more outer surfaces of the implant, such as at positions A1, A2 and V1, V2.

In another example, as illustrated in FIG. 1J, rather than a bipolar design, the system may have a mono-polar design. Unlike the bipolar system design, the mono-polar designs do not require the current carrying and measuring electrodes to be in proximity to each other. The smart implant 106 illustrated FIG. 1J includes an implant body 108 in the form of a cage, such as a lumbar interbody cage. The implant body 108 includes a bone fusion measurement system 110 with a sensor 114 suitable to measure fusion of the implant body 108 to the vertebra 130b and vertebra 130c as discussed above. The sensor 114 comprises at least one impedance electrode(s) positioned in the inner rings and on one or more outer surfaces of the implant, such as at positions A1, A2, A3. At least one conductive probe, V1, is positioned on the outer surface of a patient's skin that carried electrical current. The system may also include an antenna that enables signals to be sent and received wirelessly.

As shown, the integrated electronic circuit may be embedded inside of or on the side(s) of the cage. The bipolar electrode design will generally comprise at least one electrically conductive probe on an outer surface of the cage that carries an electrical current and is in direct contact with tissue in the area of interest and at least one electrically conductive probe that is separate from the current carrying probe which is used to measure electrical impedance. Also included is an antenna that enables signals to be sent/received wirelessly. The implant may also include additional sensors for measurement of other data such as strain, pressure, load and temperature. The probes may be configured for calibration and the data gathered may be stored for later use and/or indexing.

In addition to the electrode probes described above, an electronic circuit is also used to control the logic of current generation and measurement. The circuit may have a 2D or 3D structure and may be any suitable shape, such as circular, rectangular, oval or triangular. The PCB board may be single-sided, double-sided, flexible and or multi-sided. In addition, the implant includes an antenna so the system can obtain power wirelessly.

In various embodiments, the antenna may be attached directly to the PCB, it may be attached external to the PCB but internal to the implant or it may be attached external to both the PCB and the implant. As shown in FIG. 1K, the circuit board 250 is a long rectangular 2D plane embedded in the implant 106 and most of the electronic components 255 are located on one side while the antenna 260 is on the other side. This assembly gives the antenna a stiff backing while maintaining contact with the main board such that no additional wires are routed outside the PCB and the antenna does not require additional fixation.

As shown in FIG. 1L, the PCB 250 is shaped as in FIG. 1K and embedded in the implant 106. The antenna 260 is attached away from the PCB, preferably on a surface in the inner ring of the implant. The PCB may be printed on both sides with components 255 and a wire is run to connect the PCB and the antenna.

As shown in FIG. 1M, the PCB 250 is curved and flexible and attached on an outer surface of implant 106. The antenna 260 is attached away from the PCB, preferably on a surface in the inner ring of the implant. The PCB may be printed on both sides with components and a wire is run to connect the PCB and the antenna. The PCB and antenna may be covered by a protective layer or coating such as plastic, PEEK or titanium. The implant system may also include an external device that acts as a “reader”. The reader incorporates the following functionality: (1) it uses a safe electromagnetic field to send power and data to the implant; (2) it delivers power to the implant in a short period of time (<30 seconds); (3) the signal can reliably and consistently pass through human tissue; and (4) a data signal can be received and deciphered by the reader without additional hardware or software interface. The reader may optionally have additional functionality, such as (1) providing support for lumbar or cervical spine; (2) providing heat therapy for the area surrounding the cervical or lumbar spine; (3) provide range of motion measurements for the cervical or lumbar spine; provide electromagnetic stimulation (bone growth stimulator) to the target fusion area.

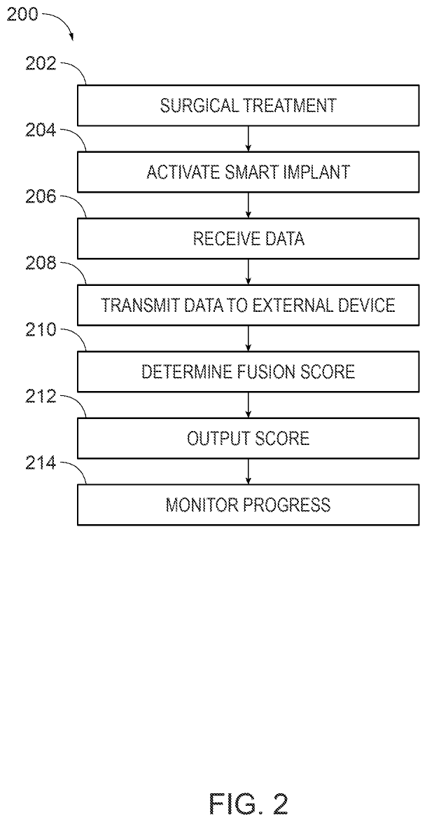

FIG. 2 illustrates a method 200 of using a smart implant 106 or wearable device 142. The operations of the method 200 may be executed in an order other than as shown. Some operations may be optional. Some operations may be executed substantially in parallel with one another.

The method 200 may begin in operation 202 and the healthcare provider 104 performs a surgical intervention on the patient 102 and implants a smart implant 106 in the body of the patient 102. For example, as discussed above with respect to FIG. 1C and FIG. 1D, the smart implant 106 may be installed on the cervical spine 136 of the patient 102. In other examples, the smart implant 106 may be installed in another portion of the body of the patient 102, such as another portion of the spine, e.g., lumbar spine. In the case of a wearable device 142, the patient 102 may be fitted with the wearable device 142.

The method 200 may proceed to operation 204 and the healthcare provider 104 activates the bone fusion measurement system 110 of the smart implant 106 or wearable device 142. For example, the healthcare provider 104 may use the external device 122 to communicate with and/or power the bone fusion measurement system 110 via the wireless interface 118 to cause the bone fusion measurement system 110 to collect health data, including bone fusion data. For example, the bone fusion measurement system 110 may be powered by the external internal power supply 120 and/or the internal power supply 120.

The method 200 may proceed to operation 206 and the sensor 114 may sense health data as described above and may transmit that data to the processing element 112 which may store and/or analyze the data and store the data/analysis on the memory component 116. For example, when the bone fusion measurement system 110 is used with a smart implant 106, the sensor 114 may measure load on the implant body 108 and may correlate that load to fusion of the bone with the implant body 108. In another example, when the bone fusion measurement system 110 is used with a wearable device 142, the sensor 114 may collect range of motion data for the patient 102. The wearable device 142 may prompt the patient 102 to make certain moves (e.g., bend neck left, bend neck right, etc.) and may record range of motion data.

The method 200 may proceed to operation 208 and the bone fusion measurement system 110 transmits the data/analysis determined in operation 206 to the external device 122. For example, the bone fusion measurement system 110 may transmit data and/or analysis to the external device 122 via the wireless interface 118 and/or input/output interface 124. The external device 122 may further transmit the data/analysis to the server 138, such as through the network 140.

The method 200 may proceed to operation 210 and a processing element 112 of the external device 122 or the server 138 further processes the data/analysis from the bone fusion measurement system 110 to determine a fusion score or rating. For example, the processing element 112 of the external device 122 and/or server 138 may interpret a digital signal generated by the analog-to-digital converter from the sensor data. The processing element 112 may process the data/analysis through a processing algorithm, for example to reduce noise, non-sensical values, false positives/negatives, or the like. The processing element 112 may apply a data analysis algorithm to determine bone fusion or other health information from the health data collected by the sensor 114. The processing element 112 may provide a user experience and/or receive user input through the input/output interface 124. The processing element 112 may determine a fusion score or rating that correlates health data with bone fusion or other health information. The processing element 112 may provide different user interfaces and/or functionality for the patient 102 and the healthcare provider 104. The processing element 112 may incorporate patient-reported outcomes (e.g., relative pain scale, dietary notes, other physiologic or psychological feedback, etc.) to develop the fusion score.

The method 200 may proceed to operation 212 and a processing element 112 of the external device 122 or the server 138 outputs the fusion score, for use by the healthcare provider 104 and/or patient 102. The score may be output by the input/output interface 124 such as via a display, email, text message, printout, or the like. The healthcare provider 104 may determine a treatment plan for the patient 102 based on the fusion score. For example, the healthcare provider 104 may prescribe exercises, rest, restricted activity, medication, or further surgical intervention based on the fusion score.

The method 200 may proceed to operation 214 and a processing element 112 of the external device 122 or the server 138 generates a healthcare provider 104 interface to enable the healthcare provider 104 to monitor patient progress. The processing element 112 may generate a patient 102 interface to allow the patient 102 to monitor their own progress and/or report outcomes (e.g., relative pain scale, dietary notes, other physiologic or psychological feedback, etc.).

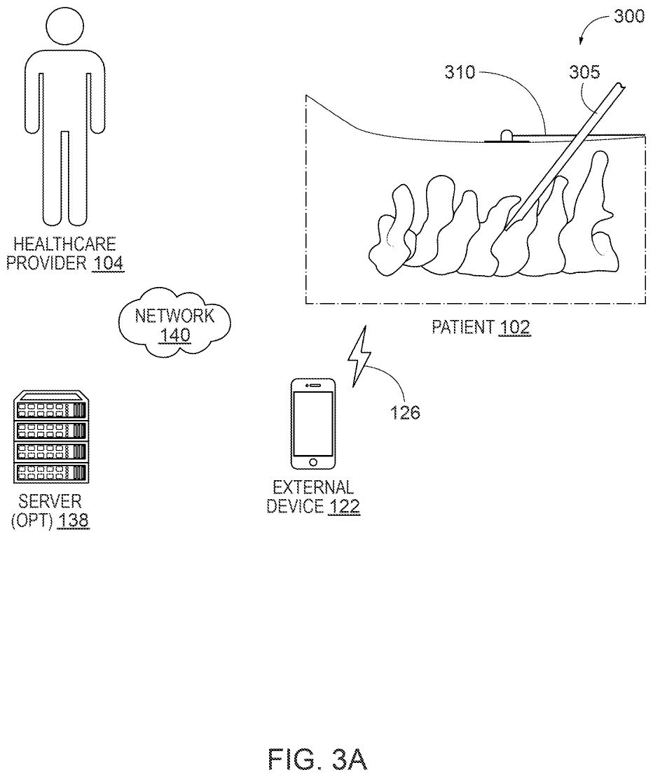

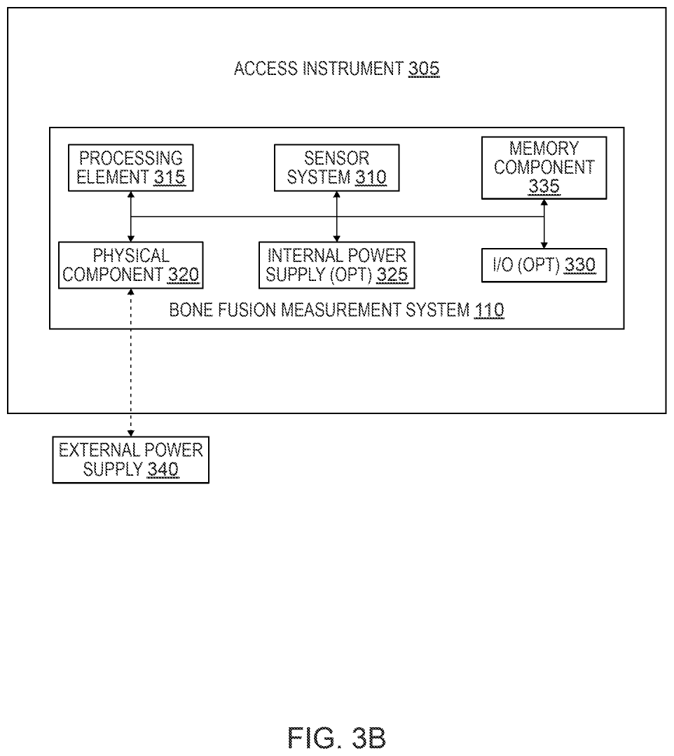

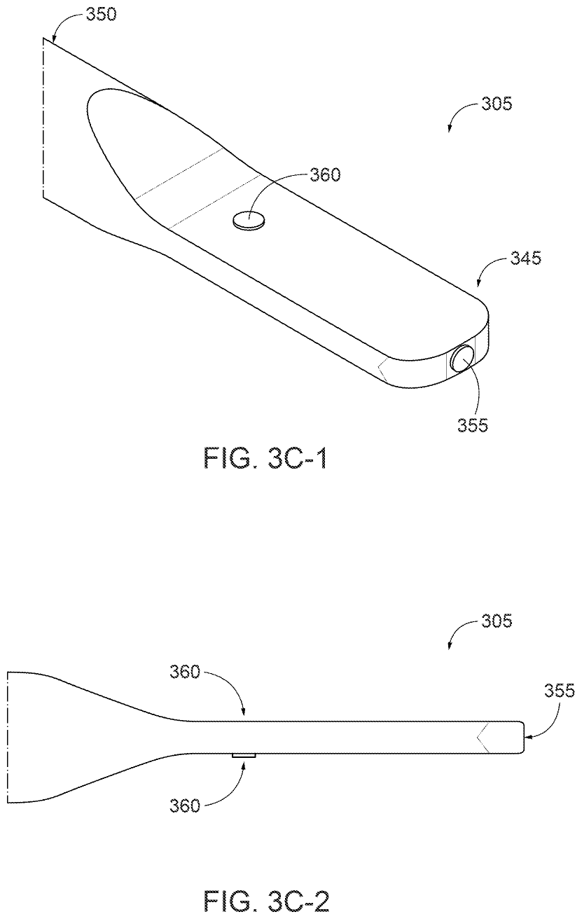

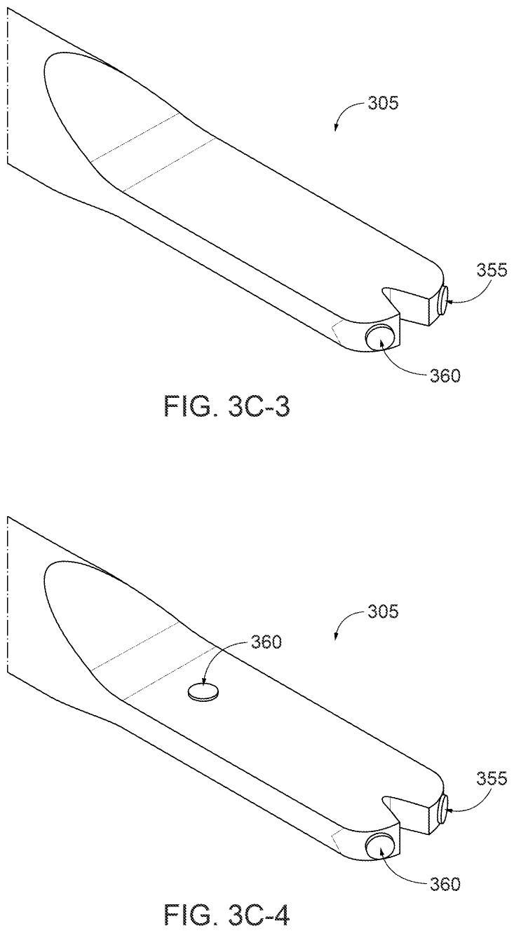

FIG. 3A is a simplified schematic representation of an access instrument system 300. The access instrument system 300 may include an access instrument 305 and the external device 122. The access instrument 305 may also be referred to herein as an access chisel 305. The access instrument 305 may be wirelessly charged via charging 126. The access instrument system 300 includes a sensor system 310 that measures health data including, but not limited to, tissue type data. The sensor system 310 may in whole or in part be integrated into the access instrument 305, but does not have to be. For example, a component of the sensor system 310 is depicted in FIG. 3A as external to the access instrument 305. The access instrument system 300 may optionally include the network 140 and/or the server 138. The network 140 and the server 138 can be the same as the network 140 and the server 138 in the bone fusion assessment system 100, but does not have to be. The healthcare provider 104 may perform the surgical intervention on the patient 102. During the intervention, the healthcare provider 104 may insert the access instrument 305 into the patient 102 and may use the access instrument 305 to locate the targeted fusion area, e.g., the area between facet joints of the patient 102, as depicted in FIG. 3A.

FIG. 3B is a simplified diagram of components of an example of an access instrument 305. The access instrument 305 may include the access instrument system 300. When the sensor system 310 is not fully integrated into the access instrument 305, e.g., a component of the sensor system 310 is external to the body of the patient 102, the access instrument system 300 may be coupled to or integrated with the component outside the body of the patient 102.

The access instrument 305 may be any device suitable to also support and/or receive the access instrument system 300. The access instrument 305 may have a generally cylindrical cross-section, such as tubular or otherwise. The access instrument 305 may include many configurations allowing at least partial insertion of the access instrument 305 into a spinal facet joint. For example, the access instrument 305 may be any device that can “dock” in cervical and lumbar facets during spinal surgery. However, the access instrument 305 may be any device for insertion into the patient 102. For example, the access instrument 305 may be a chisel, a gouge, a guide tube, or other device that provides orthopedic support, constraint, separation, or other therapeutic effect to the patient 102. See, e.g., the example access instrument 305 discussed herein with respect to FIG. 3C.

As shown in FIG. 3B, the various devices of the access instrument system 110, external device 122, and/or server 138 may include one or more processing elements 315, one or more sensor systems 310, one or more memory components 335, a physical component 320 (also referred to herein as a wireless interface 320), an optional internal power supply 325, and an optional input/output interface 330, where the various components may be in direct or indirect communication with one another, such as via one or more system buses, contract traces, wiring, or via wireless mechanisms. For example, the processing element 315 may be in communication with the sensor system 310 and the physical component 320 may be in communication with the processing element 315.

The sensor system 310 may be any system suitable to measure health data of a patient 102 such as tissue type or other tissue properties. By way of non-limiting example, the sensor system 310 may measure electrical impedance of a tissue area or relative impedance values between two or more tissue areas, e.g., soft tissue and one or more bone portions. For example, a high impedance value may indicate bone, which is one of the least conductive tissues in the body. Determining the tissue type that is in contact with a component of the sensor system 310 can help the healthcare provider 104 locate the targeted fusion area and guide the access instrument 305 into the correct position during the surgical intervention.

The sensor system 310 may use any suitable sensor technology that can measure relevant health data such as a strain sensor (e.g., a piezoelectric sensor or Wheatstone bridge strain gauge), a piezo-resistive polymer, a capacitive transducer, a passive resonator, a Surface Acoustic Wave (“SAW”) resonator, ultrasound emitter and/or detector, a galvanometer, current sensor, capacitance sensor, inductance sensor, impedance sensor, motion sensor (e.g., accelerometer), camera-based motion analyzer, or the like. Any sensor or combination of sensors may be used in the access instrument 305.

The one or more processing elements 315 may be disposed in or on the access instrument 305. The one or more processing elements 315 may be similar to or the same as the processing element 112. The one or more processing elements 315 may be substantially any electronic device capable of processing, receiving, and/or transmitting instructions. For example, the processing elements 315 may be a microprocessor, microcomputer, graphics processing unit, advanced reduced instruction set computing (“RISC”) machine (“ARM”) chip, system on a chip, or the like. The one or more processing elements 315 may be the same or different geometric structures and may be two-dimensional or three-dimensional. The processing element 315 may include an internal power supply, which may be similar to or the same as the internal power supply 325. For example, the processing element 315 may include one or more batteries or at least one capacitor. For example, the processing element 315 may include at least enough capacitors to temporarily deliver enough power to activate the sensor system 310.

The processing element 315 may be in communication with the sensor system 310. For example, the processing element 315 may send power to the sensor system 310 and may receive current data from the sensor system 310. It also should be noted that the processing elements 315 may include one or more processing elements or modules that may or may not be in communication with one another. For example, a first processing element 315 may control a first set of components of the access instrument system 300 and a second processing element 315 may control a second set of components of the access instrument system 300 where the first and second processing elements 315 may or may not be in communication with each other. Relatedly, the processing element 315 may be configured to execute one or more instructions in parallel locally, and/or across a network 140, such as through cloud computing resources such as the optional server 138.

The memory component 335 stores electronic data that may be utilized by the access instrument system 300, such as audio files, video files, document files, programming instructions, historical sensor data, chisel configuration, and the like. The memory component 335 may be, for example, non-volatile storage, a magnetic storage medium, optical storage medium, magneto-optical storage medium, read only memory, random access memory, erasable programmable memory, flash memory, or a combination of one or more types of memory components.

The physical component 320 may be referred to herein as a wireless interface 320. The physical component 320 receives and transmits data and/or power to and from the access instrument system 300 and the external device 122 or other devices of the access instrument system 300, such as a second processing element. See, e.g., the wireless communication/charging 126 shown schematically in FIG. 3A. For example, the physical component 320 may be in communication with the processing element 315. The physical component 320 may receive the impedance measurement from the processing element 315 and the physical component 320 may also transmit the impedance measurement, as discussed herein.

The physical component 320 may transmit data, such as the impedance measurement, to the external device 122 directly or indirectly. For example, the physical component 320 may transmit data to and from other computing devices through the network 140 or directly from device to device. In some embodiments, the physical component 320 may also include various modules, such as an application program interface (API) that interfaces and translates requests across the network 140 to external device 122. The physical component 320 may be any suitable wired or wireless interface. For example, the physical component 320 may include Wi-Fi, Bluetooth, Wi-Max, Zigbee, near field communication (“NFC”), radio frequency identification (“RFID”), a passive integrated transponder (“PIT”), cellular data, or the like.

The physical component 320 may be any shape. For example, the physical component 320 may two-dimensional or three-dimensional geometric shapes, such as a rectangular or a circular pattern. The physical component 320 may be communicatively coupled with the processing element 315. The physical component 320 may be directly attached to the processing element 315, external to the processing element 315 and internal to the access instrument 305, and/or external to the processing element 315 and external to the access instrument 305. The processing element 315 and/or the physical component 320 may be protected by an additional layer of material, such as plastic, PEEK, or titanium.

The physical component 320 may provide either, or both, data transmission/receipt and power to the access instrument system 300. For example, the physical component 320 may transmit the impedance measurement to the external device 122, also referred to herein as a second processing element 122. The second processing element 122 may store impedance values and/or measurements and calculate a difference between and/or in the stored impedance values and/or measurements.

Additionally, the physical component 320 may be or may include an antenna that receives electromagnetic energy from the external device 122 used to power the access instrument system 300 without the access instrument system 300 using an internal power supply 325, or the electromagnetic energy from the external device 122 may be used to charge the internal power supply 325. For example, the physical component 320 may transmit power from the external device 122 to the processing element 315. For example, the external device 122 may include an external power supply 340 such as a radio frequency or magnetic induction antenna or coil that induces the flow of an electric current in the physical component 320 when the external power supply 340 and the physical component 320 are in proximity to one another. In some examples, the external device 122 is electromagnetic such that power may be transferred between the physical component 320 and the external device 122 by a magnetic field using inductive coupling (e.g., resonant inductive coupling) between coils of conductors in the physical component 320 and the external device 122, such as when the physical component 320 supports RFID. For example, the external power supply 340 may generate a time-varying electromagnetic field, which transmits power across space to the physical component 320, which extracts power from the electromagnetic field and supplies it to an electrical load in the access instrument system 300. In some examples, power may be transferred by using electric fields using capacitive coupling between conductive electrodes. In some examples, the external power supply 340 may transmit power to the physical component 320 via magnetodynamic coupling via one or more rotating magnets, microwaves, visible light waves, or other portions of the electromagnetic spectrum, etc. In some embodiments, separate external devices 122 may be used to separately power the access instrument system 300 and transmit/receive data. In some examples, the external device 122 may be a smart phone, smart watch, tablet computer, or the like. In some examples, data transmission between the external device 122 and the access instrument system 300 may be encrypted, such as to protect patient privacy.

The physical component 320 may receive power from the external power supply 340 and transmit data to the external device 122 simultaneously or in series. In some implementations, the external power supply 340 may provide power to the access instrument system 300 via the physical component 320 and the access instrument system 300 may charge the internal power supply 325 if used. Thus, the access instrument system 300 may be able to measure sensor data and/or store sensor data when the external power supply 340 is not present.

The various devices of the system may optionally include the internal power supply 325, as discussed herein. The internal power supply 325 provides power to various components of the access instrument system 300 or the external device 122. The internal power supply 325 may include one or more rechargeable, disposable, or hardwire sources, e.g., batteries, capacitor, dual layer capacitor (i.e., super capacitor) power cord, AC/DC inverter, DC/DC converter, or the like. In some embodiments, where the internal power supply 325 is a battery, the battery may be a rechargeable (i.e., secondary) battery or may be a single-use non-rechargeable (primary) battery. When a primary battery is used, the battery life may be long enough for the useful life of the access instrument 305. Additionally, the internal power supply 325 may include one or more types of connectors or components that provide different types of power to the access instrument system 300. In some embodiments, the internal power supply 325 may include a connector (such as a universal serial bus) that provides power to the access instrument system 300 or batteries access instrument system 300 or external device 122 and also transmits data to and from the device to other devices.

The optional input/output interface 330 allows the access instrument system 300 and/or external device 122 to receive input from a user and provide output to a user. For example, the input/output interface 330 may include a capacitive touch screen, keyboard, mouse, stylus, button, knob or the like. The type of devices that interact via the input/output interface 330 may be varied as desired. The input/output interface 330 provides an input/output mechanism for devices of the access instrument system 300, such as to display visual information (e.g., images, graphical user interfaces, videos, notifications, and the like) to a user such as a healthcare provider 104, and in certain instances may also act to receive user input (e.g., via a touch screen or the like). The display may be an LCD screen, plasma screen, LED screen, an organic LED screen, or the like. The type and number of displays may vary with the type of devices (e.g., smartphone versus a desktop computer). In some example, the I/O interface 330 may include an interface to the network 140, such as Wi-Fi, Bluetooth, an Ethernet socket, or the like. In some embodiments, data transmission from the sensor system 310 to the external device 122 may be via the input/output interface 330 instead of, or in addition to, the physical component 320. For example, the external device 122 may communicate to the access instrument system 300 via wired communication, e.g., a device connected to the skin surface of the patient 102.

The access instrument system 300 may also include an analog to digital conversion device, which may be part of the sensor system 310, processing element 315, or may be a separate device. The analog to digital conversion device may receive an analog signal from a sensing element in the sensor system 310 and convert the same to a digital signal for further processing by the processing element 315, storage in the memory component 335, and/or communication to the external device 122 via the physical component 320. For example, the physical component 320 may transmit the impedance measurement to the second processing element.

The processing element 315 may execute an algorithm or firmware (stored for example on the memory component 335), to reject over-sensitivity, false positives, errors, or excess information received from the sensor system 310. The access instrument system 300 may have a novel layout allowing for miniaturization including components like antennas, capacitors, flexible circuit boards, other novel design and layouts.

FIG. 3C-1 is a simplified perspective view of an example of the access instrument 305. The access instrument 305 may include or define a distal end 345 and a proximal end 350. The proximal end 350 may be arranged to facilitate user (e.g., the healthcare provider 104) manipulation of the access instrument 305. For instance, the proximal end 350 may be generally solid with an end surface for malleting the access instrument 305 into position. In some examples, the proximal end 350 may include an alignment mark. For example, a groove may be defined on the proximal end 350 at a distance away from distal end 345. The groove may extend circumferentially around the proximal end 350. As explained below, the alignment mark and/or groove may align with a corresponding alignment feature of another tool or instrument to define a seated position of the access instrument 305 and/or the other tool or instrument.

The distal end 345 may include or define a pointed and/or chiseled tip for insertion into the patient 102, e.g., after a small incision is made. For example, the chiseled tip may wedge inside a facet joint. The chiseled tip may by chamfered such that the access instrument 305 may be driven into and/or otherwise anchored in the spinal facet joint. Depending on the application, the chiseled tip may have one or more chamfers, such as a single chamfer, a double chamfer, or more than two chamfers.

The distal end 345 and/or the chiseled tip may be or may include a first portion and a second portion. The ends of the first and second portions of the distal end 345 may be separated from each other by an air gap (see e.g. FIGS. 3C-3). The access instrument 305 may include at least one button, knob, or similar mechanical device to alter a frequency of the current sent by the current carrying electrode 355. For example, the knob or button may influence or otherwise control the amount of power sent by the processing element 315 to the sensor system 310. The access instrument 305 may be any material suitable to host the sensor system 310. For example, the access instrument 305 may be three-dimensionally printed in an insulating material. Additionally, the access instrument 305 may be plastic and the chiseled tip of the distal end 345 of the access instrument 305 may comprise an insulating material such as stainless steel, titanium, or the like.

The sensor system 310 may include at least one pair of electrode probes. The electrode probes may be integrated into or onto the access instrument 305, but do not have to be. For example, one of the electrode probes of the pair of electrode probes may contact a skin surface and the other of the electrode probes of the pair of electrode probes may contact a tissue area such as bone. The electrode probes may both contact a tissue area.

The electrode probes may include at least one current carrying electrode 355 and at least one current measuring electrode 360. The current carrying electrode 355 and the current measuring electrode 360 may be or function similar to or the same as the current carrying electrode and the current measuring electrode in the sensors 114. For example, the current carrying electrode 355 may receive power sent by the processing element 315 and may send current to a tissue area, such as bone. The current measuring electrode 360 may receive current from the tissue area and may generate current data. Thus, the current measuring electrode 360 may be separated from the current carrying electrode 355, and visa-versa.

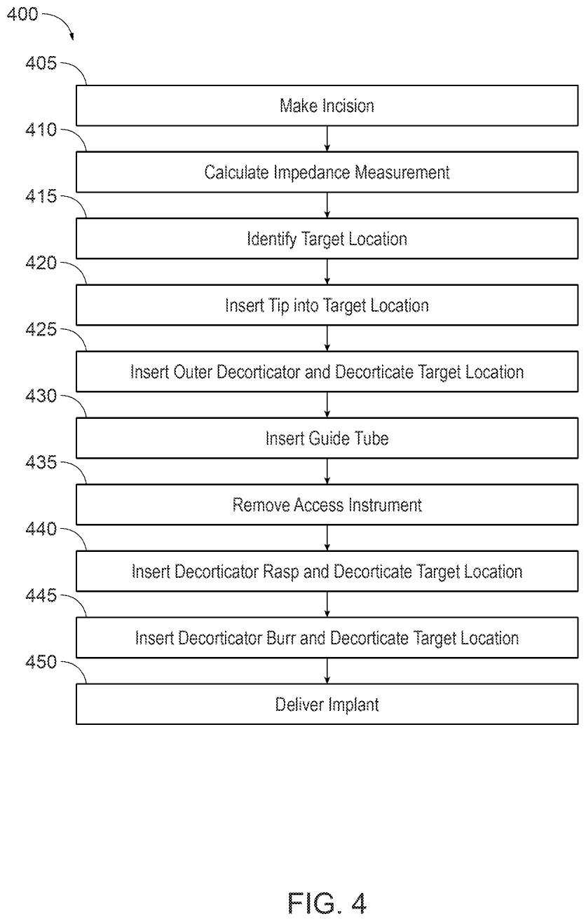

The layout of the electrodes 355, 360 can vary, thus the electrodes 355, 360 may be oriented in any manner suitable for current generation and measurement. For example, the current carrying electrode 355 may be disposed on or at the distal end 345 of the access instrument 305 and may be in direct contact with the tissue area. In a bi-polar design, such as the embodiment shown in FIG. 3C-3, the current measuring electrode 360 may be separated from the current carrying electrode 355 by an air gap, e.g., the current carrying electrode 355 may be disposed on the first portion of the distal end 345 and the current measuring electrode 360 may be disposed on the second portion of the distal end 345, or vice-versa. Alternatively, in a mono-polar design, such as the embodiment shown in FIG. 3C-4, the current measuring electrode 360 may be separated from the current carrying electrode 355 by insulation. For example, the current carrying electrode 355 may be disposed at the distal end and the current measuring electrode 360 may be disposed a distance towards the proximal end 350 of the access instrument 305. For example, the current measuring electrode 360 may be disposed around 3 centimeters (+/−10%) towards the proximal end 350 from the current carrying electrode 355. Further, in a mono/bi-polar hybrid design, the current carrying electrode 355 may be encased in an insulating sleeve and the current measuring electrode 360 may encase the insulating sleeve.

The current carrying electrode 355 and the current measuring electrode 360 may both be disposed on the or at the chiseled tip of the distal end 345. For example, the current carrying electrode 355 may be disposed at the distal end 345 and a pair of current measuring electrodes 360 may be disposed at a distance towards the proximal end 350 of the of the access instrument 305 (but still on the chiseled tip of the distal end 345), and on opposite surfaces of the access instrument 305. In this example, since all the electrodes 355, 360 are disposed on or at the chiseled tip and the chiseled tip may insert into a facet joint of the patient 102, the pair of current measuring electrodes 360 may obtain the current data for a first tissue area above the access instrument 305 and obtain the current data for a second tissue area below the access instrument 305 (e.g., in an operational orientation of the access instrument 305).

Thus, a first impedance measurement may be calculated for the first tissue area and a second impedance measurement may be calculated for the second tissue area. The first impedance measurement may be different from the second impedance measurement, which may indicate the chiseled tip of the access instrument 305 is not positioned in a facet. The first impedance measurement may be similar to or the same as the second impedance measurement, which may indicate that each of the opposite surfaces of the access instrument 305 are in contact with a boney tissue and the chiseled tip of the access instrument 305 positioned in the facet. The pair of current measuring electrodes 360 may be disposed on any of the surfaces, ends (e.g., the distal end 345), or sides of the access instrument 305. For example, there may be the electrodes 355, 360 each disposed at the distal end 345 to obtain current data to identify the tissue type of the tissue area at the initial point of contact with the access instrument 305. In another example, there may be a plurality of current measuring electrodes 360, and at least one current measuring electrode 360 of the plurality may be on each surface of the access instrument 305 to obtain the current data for a plurality of tissue areas surrounding the access instrument 305.

The current carrying electrode 355 does not need to be disposed at the distal end 345 of the access instrument 305 or in direct contact with the tissue area. For example, the current carrying electrode 355 may not be physically coupled with the access instrument and may be external such that the current carrying electrode 355 may contact a skin surface of the patient 102. In this example, the current measuring electrode 360 may be disposed at the distal end 345 of the access instrument 305.

There may be more than one pair of electrodes. For example, the current carrying electrode 355 may be disposed at the distal end 345 of the access instrument 305 and a pair of current measuring electrodes 360 may be disposed between the proximal end 350 and the distal end 345 of the access instrument 305. Additionally, a first current carrying electrode may be disposed at the first portion of the distal end 345 of the access instrument 305 and a first current measuring electrode may be disposed at the second portion of the distal end 345 of the access instrument 305 with a second current carrying electrode and a second current measuring electrode disposed between the proximal end 350 and the distal end 345 of access instrument 305. The second current measuring electrode separated from the second current carrying electrode.

The processing element 315 may calculate an impedance measurement based on the current data and the power sent to the sensor system 310. For example, the processing element 315 may send power to the sensor system 310 and the current carrying electrode 355 may receive the power sent by the processing element 315 and may send current to the tissue area. The current measuring electrode 360 may receive current from the tissue area and may generate current data. The processing element 315 may receive the current data from the current measuring electrode 360 and may calculate an impedance measurement corresponding to the current sent from the current carrying electrode 355 and the current received by current measuring electrode 360.

FIG. 4 illustrates a method 400 of delivering an implant, such as the smart implant 106. The implant may be a lumbar cage, a cervical spine cage, or a similar implant. The implant may be one of a lumbar cage and a cervical spine cage adapted to be similar to or the same as the smart implant 106. In block 405, the method 400 may include making an incision. Making an incision may include exposing target bony elements. The healthcare provider 104 may make an incision using any appropriate tool or device.

In block 410, the method 400 may include calculating an impedance measurement or a plurality of impedance measurements. The plurality of impedance measurements may be calculated via a processing element in communication with a sensor system of an access instrument. The access instrument may be similar to or the same as the access instrument 305 described herein. The sensor system may be similar to or the same as the sensor system 310 described herein. The processing element may be similar to or the same as the processing element 315 described herein. For example, the plurality of impedance measurements may be calculated similarly to or the same as the impedance measurement calculations described herein to identify tissue type of tissue areas surrounding the access instrument.

In block 415, the method 400 may include identifying a target location based on the plurality of impedance measurements. Identifying the target location may include calculating a difference in the plurality of impedance measurements, similarly to or the same as previously described. Identifying the target location may include identifying a tissue type based on the difference, similarly to or the same as previously described. The tissue type may be muscles, tendons, ligaments, connective tissue such as fascia, and bone. The target location may include tissue types such as muscles, tendons, ligaments, connective tissue such as fascia, and bone. For example, the target location may be a facet joint.

In block 420, the method 400 may include inserting a tip of an access instrument into the target location. The access instrument may be similar to or the same as the access instrument 305 described herein. For example, the tip of the access instrument may wedge inside or between the facet joint. The sensor system may be used to locate the target location. For example, the current data obtained by the sensor system may be used to identify boney tissue on each side or surface of the access instrument 305 to indicate the tip of the access instrument 305 is positioned in the target location, e.g., in the facet joint.

In block 425, the method 400 may include inserting an outer decorticator over the access instrument and decorticating the target location using the outer decorticator. The outer decorticator may be slidably inserted over the access instrument. The outer decorticator may include a proximal portion formed to engage a handle or otherwise allow a practitioner to directly or indirectly (such as through a robotic arm) grasp the proximal portion to rotate or otherwise operate the outer decorticator. A tubular shaft may connect the proximal portion to a distal portion. The distal portion may be shaped with a decorticator or rough surface that may be used to roughen or abrade the surface of the target location. In some examples, the shaft of the outer decorticator is hollow, with a larger inner diameter than an outer diameter of the access instrument 305. Decorticating the target location may include decorticating superior lateral masses and/or inferior lateral masses of the target location. The method 400 may include slidably removing the outer decorticator while leaving the access instrument positioned in the target location.

In block 430, the method may include inserting a guide tube over the access instrument. The guide tube may include a tubular shaft to connect a proximal portion that is connected to forks formed at the distal end of the guide tube. The shaft may be hollow, with an inner diameter that is larger than the outer diameter of the access instrument 305. The method 400 may include positioning forks of the guide tube adjacent an outside of a tip of the access instrument. The guide tube to may hold vertebrae apart. In block 435, the method may include removing the access instrument 305 through the shaft of the guide tube. For example, the guide tube may slide over the access instrument 305 to position the forks within the target location, e.g., the facet joint. Once the forks are positioned, the access instrument 305 may be removed by sliding the chiseled tip and the access instrument 305 through the hollow shaft of the guide tube.

In block 440, the method may include inserting a decorticator rasp through the shaft of the guide tube and decorticating the target location using the decorticator rasp. Decorticating the target location may include decorticating articular surfaces of the target location. The method 400 may include slidably removing the decorticator rasp through the guide tube.

In block 445, the method 400 may include inserting a decorticator burr through the guide tube. The decorticator burr may include a proximal portion, a burred end formed at a distal portion opposite the proximal end, and a tubular shaft connecting the proximal portion and burred end. The outer diameter of the shaft may be smaller than the inner diameter of the guide tube shaft. The decorticator burr may be slidably inserted through the guide tube. For example, the burred end of the decorticator burr may be inserted into the proximal portion of the guide tube, and slid through the shaft until it extends between and/or past the forks of the guide tube. The method 400 may include decorticating the target location using the decorticator burr. Decorticating the target location may include decorticating articular surfaces of the target location. The method 400 may include slidably removing the decorticator burr through the guide tube.

In block 450, the method 400 may include delivering the implant, e.g., the smart implant 106. For example, the method 400 may include applying spinal instrumentation as appropriate. The spinal instrumentation may include, but is not limited to applying bone graft, inserting an implant, or various combinations thereof. The method 400 may include activating the implant, e.g., the sensor system of the implant, similarly to or the same as described herein.

The delivery system or apparatus disclosed herein is advantageous for at least the following reasons. First, the system facilitates delivery of an implant to a facet joint via a minimally invasive or percutaneous procedure, reducing the risk, surgical time and recovery time associated with the implantation of the implant in the facet joint. Accordingly, many of the dimensional characteristics associated with the delivery system, its components, and the implant are advantageous in that they facilitate or make possible the minimally invasive or percutaneous procedures described herein. Second, the system may facilitate the implant being delivered while the patient is capable of providing verbal feedback as to the impact of the implant relative to symptoms being felt by the patient.

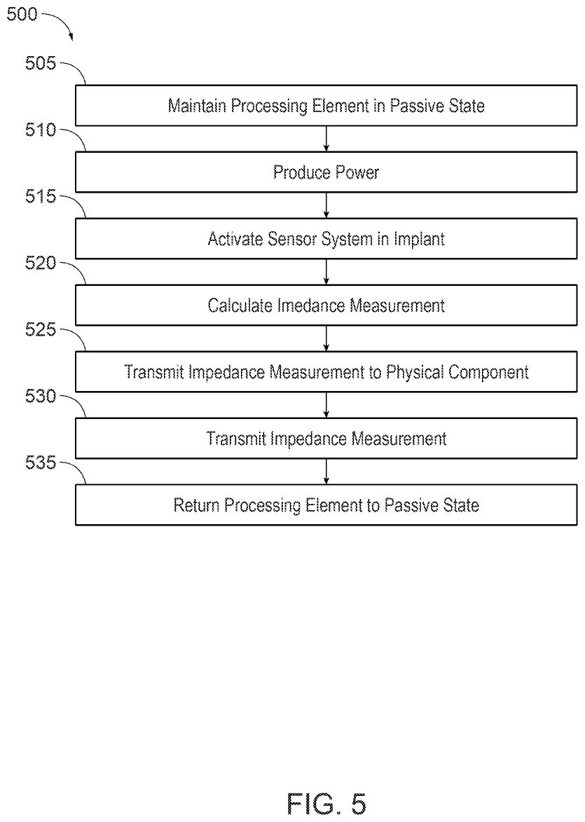

FIG. 5 illustrates a method 500 of activating an implant, such as the smart implant 106. The implant may be a lumbar cage, a cervical spine cage, or a similar implant. The implant may be one of a lumbar cage and a cervical spine cage adapted to be similar to or the same as the smart implant 106. In block 505, the method 500 may include maintaining a processing element in a passive or semi-passive state. For example, the implant may include an internal power supply, e.g., the internal power supply 120. The internal power supply may include only enough capacitors to temporarily deliver power to activate a sensor system and/or a sensor, e.g., the sensor 114, to make a single reading before the capacitors discharge completely. Thus, when the sensor is not activated, the implant is maintained in a passive or semi-passive state. The processing element may be similar to or the same as processing elements 112. For example, the processing element 112 may be disposed in or on the implant.

In block 510, the method 500 may include producing power. For example, the internal power supply may produce power. Similar to the internal power supply 120, the internal power supply may include one or more rechargeable, disposable, or hardwire sources, e.g., batteries, capacitor, dual layer capacitor (i.e., super capacitor) power cord, AC/DC inverter, DC/DC converter, or the like. In some embodiments, where the internal power supply is a battery, the battery may be a rechargeable (i.e., secondary) battery or may be a single-use non-rechargeable (primary) battery. When a primary battery is used, the battery life may be long enough for the useful life of the implant. Additionally, the internal power supply may include one or more types of connectors or components that provide different types of power. In some embodiments, an external power supply, e.g., the external power supply 134, may provide power via a wireless interface, e.g., wireless interface 118, to charge the internal power supply if used. For example, the external device may deliver power to the passive implant within a certain amount of time (e.g. about 30 seconds). In block 515, the method 500 may include activating a sensor system in the implant. For example, the sensor system may receive power produced by the internal power supply and delivered by the processing element. The sensor system may be similar to or the same as the sensor systems described herein. The sensor system may be or may include sensors similar to or the same as sensors described herein, e.g., the sensor 114. For example, the sensor may be any device suitable to measure health data of a patient such as bone fusion or other bone properties. By way of non-limiting example, the sensor may measure motion (e.g., micro-motion, flex, looseness) of the implant or relative motion between the implant and/or one or more bone portions. The sensor may measure motion relative to a reference point (e.g., a surface of a joint). By way of non-limiting example, the sensor may be used to measure electrical properties such as Charge (Q), Capacitance (C), Inductance (L), Voltage (V), Current (I), Resistance (omega), Power (P), Conductance (G), Impedance (Z), Frequency (f), and Amplitude (A). With one, some, or any of these properties being measured, it is possible to interpret a change in the circuit and thus, a change in fusion. It is also noted that a device that has any electrical properties, also can create a magnetic field, for example, pulses of magnetism, that can be sensed and turned back into an electrical signal, a property called inductance.

For example, and with reference to FIGS. 1K/1L, the pressure sensor is using a physical force to change or create an electrical signal, which varies it's V, I, R, O, Z, G, I, C, etc. electrical property, and any of those properties can be read and interpreted (based on the change from the original signal), to determine a change, in this case, pressure. Looking at FIGS. 1K/1L, those sensors could be measuring light (more bone=less light), pressure (more bone+more/less pressure), resistance (bone has different resistance than blood/air), but all these examples require the signal to be simplified to basic electrical or magnetic properties like (V, I, R, f, Z, etc. . . . ) to be transmitted to an external device.

Additionally, the sensor system may include a current carrying electrode and a current measuring electrode. For example, the current carrying electrode may receive power produced by the internal power supply and sent by the processing element. The current carrying electrode may send current to a tissue area. The current measuring electrode may receive current from the tissue area and may generate data or current data. The current measuring electrode may send the current data to the processing element. The current measuring electrode may be separated from the current carrying electrode, and vice-versa. For example, the current carrying electrode may be disposed on the outer surface of the implant and may be in direct contact with the tissue area, and the current measuring electrode may be separated from the current carrying electrode by insulation or an air gap. Further, the current carrying electrode does not need to be disposed in or on the implant. For example, the current carrying electrode may contact a skin surface and the current measuring electrode may be disposed on the outer surface of the implant. The current carrying electrode and the current measuring electrode may be disposed on any outer surface of the implant, e.g., the top surface, the bottom surface, the side surfaces, and/or the through surface and outer side surface in the case the implant is a cage that defines the opening.

In block 520, the method 500 may include calculating an impedance measurement. For example, the processing element may calculate the impedance measurement. The impedance measurement may be based on the current data sent by the current measuring electrode and the power produced by the internal power supply. For example, the impedance measurement may correspond to the current sent from the current carrying electrode and the current received by the current measuring electrode. For example, the processing element may send power produced by the internal power supply to the sensor system and the current carrying electrode may receive the power sent by the processing element and may send current to the tissue area. The current measuring electrode may receive current from the tissue area and may generate current data. The processing element may receive the current data from the current measuring electrode and may calculate the impedance measurement corresponding to the current sent from the current carrying electrode and the current received by current measuring electrode.

In block 525, the method 500 may include transmitting the impedance measurement. For example, the method 500 may include transmitting the impedance measurement to a physical component of the implant. The physical component may be the same as or similar to the wireless interface 118, which is also referred to herein as the physical component 118. For example, the processing element, internal power supply, and the physical component may all be communicably coupled with each other. The physical component may receive data from the implant via the processing element. The physical component may be any shape. For example, the physical component may two-dimensional or three-dimensional geometric shapes, such as a rectangular or a circular pattern. The physical component may be directly attached to the processing element, external to the processing element and internal to the implant, and/or external to the processing element and external to the implant.

In block 530, the method 500 may include transmitting the impedance measurement. For example, the method 500 may include transmitting the impedance measurement to an external device via the physical component. The physical component may receive and transmit data and/or power to and from the implant and other devices associated with the implant, such as a second processing element of the external device. For example, the physical component may receive the impedance measurement from the processing element and the physical component may also transmit the impedance measurement to the second processing element. The physical component may transmit data, such as the impedance measurement, to the external device directly or indirectly, similarly as described herein. Additionally, the external device may deliver power to the physical component. For example, the external device may be or may include an electromagnetic power source. The physical component may charge the capacitors in the internal power supply with the power delivered by the external device.

In block 535, the method 500 may include returning the processing element to the passive or semi-passive state. As described above, the implant may include the internal power supply with only enough capacitors to temporarily deliver power to activate the sensor system to make a single reading before the capacitors discharge completely. Thus, after activation of the sensor system, the processing element may return to the passive or semi-passive state. Additionally, the processing element may return to the passive or semi-passive state after the transmittal of the current data from the sensor system and/or after the transmittal of the impedance measurement from the physical component.

Not every method step described with reference to FIGS. 2, 4, and 5 must be included. Additionally, the method steps may be performed out of order.

The description of certain embodiments included herein is merely exemplary in nature and is in no way intended to limit the scope of the disclosure or its applications or uses. In the included detailed description of embodiments of the present systems and methods, reference is made to the accompanying drawings which form a part hereof, and which are shown by way of illustration specific to embodiments in which the described systems and methods may be practiced. These embodiments are described in sufficient detail to enable those skilled in the art to practice presently disclosed systems and methods, and it is to be understood that other embodiments may be utilized, and that structural and logical changes may be made without departing from the spirit and scope of the disclosure. Moreover, for the purpose of clarity, detailed descriptions of certain features will not be discussed when they would be apparent to those with skill in the art so as not to obscure the description of embodiments of the disclosure. The included detailed description is therefore not to be taken in a limiting sense, and the scope of the disclosure is defined only by the appended claims.

From the foregoing it will be appreciated that, although specific embodiments of the invention have been described herein for purposes of illustration, various modifications may be made without deviating from the spirit and scope of the invention.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1. A bone fusion assessment system comprising:
a smart implant including:
an implant body adapted to engage with or be affixed to a bone of a patient and to provide a therapeutic benefit thereto;
a bone fusion measurement system including:
a processing element,
a sensor in electrical communication with the processing element and adapted to measure health data of the patient, and
a wireless interface adapted to receive power and transmit the health data.

2. The bone fusion assessment system of claim 1, wherein the health data includes a physical characteristic selected from voltage, resistance and impedance which is interpreted to provide bone fusion data relating to fusion of the implant body to the bone of the patient, and the sensor is adapted to measure the bone fusion data.

3. The bone fusion assessment system of claim 1, further comprising an external device adapted to transmit the power to the bone fusion measurement system via the wireless interface.

4. The bone fusion assessment system of claim 3, wherein the external device is further adapted to receive the health data via the wireless interface.

5. The bone fusion assessment system of claim 3, wherein the bone fusion measurement system includes an internal power supply adapted to be charged from the external device via the wireless interface.

6. The bone fusion assessment system of claim 3, wherein the bone fusion measurement system includes an internal power supply comprising a primary battery.

7. The bone fusion assessment system of claim 1, wherein the sensor comprises at least one of a strain sensor, a piezo-resistive polymer, a capacitive transducer, a passive resonator, a surface acoustic wave resonator, an ultrasound emitter and detector, a galvanometer, a current sensor, a capacitance sensor, an inductance sensor, or an impedance sensor.

8. The bone fusion assessment system of claim 7, wherein the strain sensor includes at least one of a piezoelectric sensor or Wheatstone bridge strain gauge.

9. The bone fusion assessment system of claim 2, wherein the bone fusion data includes at least one of bone density, bone growth, a temperature change, an electrical property, a bone loading.

10. The bone fusion assessment system of claim 9, wherein the bone loading includes at least one of a force, stress, strain, or pressure.

11. A bone fusion assessment system comprising:
a wearable device adapted to be worn by a patient, wherein the wearable device includes a bone fusion measurement system including:
a processing element,
a sensor in electrical communication with the processing element and adapted to measure health data of the patient, and
a wireless interface adapted to receive power and transmit the health data.

12. The bone fusion assessment system of claim 11, wherein the sensor includes at least one of a motion sensor, camera-based motion analyzer, a strain gauge, an impedance sensor, or an ultrasound transmitter/receiver.

13. The bone fusion assessment system of claim 12, wherein the motion sensor comprises an accelerometer.

14. The bone fusion assessment system of claim 11, wherein the health data includes at least one of a gait, a range of motion, excessive motion, a loading on the wearable device, a bone growth, or a mobility of the patient.

15. A method of assessing bone fusion of a patient comprising:
supplying at least one of a smart implant or a wearable device to a patient, wherein the smart implant or wearable device includes:
an implant body adapted to be affixed to a bone of a patient and to provide a therapeutic benefit thereto;
a bone fusion measurement system including:
a processing element,
a sensor in electrical communication with the processing element and adapted to measure health data of the patient, and
a wireless interface adapted to receive power and transmit the health data.

16. The method of claim 15, further comprising powering the bone fusion measurement system via the wireless interface to cause the bone fusion measurement system to measure the health data.

17. The method of claim 16, further comprising receiving from the bone fusion measurement system the health data by an external device.

18. The method of claim 17, further comprising determining by the external device a fusion score based on the health data,
displaying the fusion score to a healthcare provider; and
determining a treatment plan based on the fusion score.

19. (canceled)

20. The method of claim 15, wherein the health data includes bone fusion data relating to fusion of the implant body to the bone of the patient, and the sensor is adapted to measure the bone fusion data.

21. The method of claim 15, wherein the sensor comprises at least one of a strain sensor, a piezo-resistive polymer, a capacitive transducer, a passive resonator, a surface acoustic wave resonator, an ultrasound emitter and detector, a galvanometer, a current sensor, a capacitance sensor, an inductance sensor, an impedance sensor, a motion sensor, or a camera-based motion analyzer.

22.-64. (canceled)
